(19) United States
(12) Patent Application Publication
(10) Pub. No.: US 2002/0156016 A1
(43) Pub. Date: Oct. 24, 2002

(54) Control of cell growth by altering cell membrane potentials

(76) Inventor: Gerald Minuk, Manitoba (CA)

Correspondence Address: ADE & COMPANY, 1700-360 MAIN STREET, WINNIPEG, MB, R3C3Z3, CA

(21) Appl. No.: 10/114,009

(22) Filed: Mar. 28, 2002

Related U.S. Application Data
(60) Provisional application No. 60/279,739, filed on Mar. 30, 2001.
(60) Provisional application No. 60/290,347, filed on May 14, 2001.

(51) Int. Cl. A61K 38/17; A61K 31/5513; A61K 31/55; A61K 31/513; A61K 31/496; A61K 31/19

(52) U.S. Cl. 514/12; 514/221; 514/217; 514/253.08; 514/312; 514/271; 514/305; 514/557; 514/422

(57) ABSTRACT

Gamma aminobutyric acid (GABA) is a potent inhibitory neurotransmitter that binds to hetero-oligomeric receptors in the mammalian brain. In a previous study, we documented specific GABA binding to isolated rat hepatocytes which resulted in inhibition of hepatocyte proliferation. The purpose of the present study was to define the nature of hepatic GABA_{A} receptors and document their expression during rapid liver growth (post-partial hepatectomy). Polymerase chain reactions (PCR) with gene-specific primers derived from published sequences were performed with marathon-ready human and rat liver cDNA. Two GABA_{A} receptor subunit types (β3 and ε) were expressed in the human liver and one (β3) in the rat liver. PCR amplification of the human GABA_{A} receptor β3 subunit produced a single product (m.w. 53-59 kDa). In the case of the ε subunit, two PCR products were identified. Following partial hepatectomy, GABA_{A} receptor β3 subunit expression inversely correlated with regenerative activity (r=−0.527, p=0.006). In conclusion, these results indicate that in the human liver, GABA_{A} receptors consist of the β3 and ε subunit types whereas in the rat liver, only the β3 subunit type is expressed. The results also support the hypothesis that GABAergic activity serves to maintain hepatocytes in a quiescent state.

This application claims priority under 35 USC §119(e) to Provisional Patent Application Serial No. 60/279,739, filed Mar. 30, 2001 and Provisional Patent Application Serial No. 60/290,347, filed on May 14, 2001.

### FIELD OF THE INVENTION

The present invention relates generally to the field of the regulation of cell growth.

### BACKGROUND OF THE INVENTION

Hepatocellular carcinoma (HCC) is one of the most common, lethal cancers in young, adult males. Worldwide, the estimated incidence of this tumor is 1,000,000 cases per year (London, 1981, Hum Pathol 12: 1085-1097). In endemic areas, the prevalence is approximately 150 cases per 100,000 population (Di Bisceglie et al., 1988, Ann Intern Med 108: 390-401). Although less common in North America and Europe, increased immigration of hepatitis B and C carriers from developing nations and the advancing stage of liver disease in individuals with parentally acquired viral hepatitis in the 1960's and 70's has led to significant increases in the number of HCC cases being diagnosed over the past 10 years. Unfortunately, the therapeutic options presently available to treat HCC are limited. Indeed, surgical restriction remains the only potentially curative form of treatment and this option is often precluded by the advanced stage of the tumor at the time of diagnosis (Hobbs and Dusheiko, 1992, J Hepatol 15: 281-283; Okuda, 1992, Hepatology 15: 948-963).

### SUMMARY OF THE INVENTION

According to a first aspect of the invention, there is provided a method of regulating cell growth comprising:

administering an effective amount of a membrane potential regulating agent to a tissue portion.

According to a second aspect of the invention, there is provided a method of treating a patient with cancer, comprising introducing into the patient an expression vector encoding GABA receptor, such that an amount of GABA receptor effective to hyperpolarize malignant cells is expressed in the patient.

According to a third aspect of the invention, there is provided a method of treating cancer in a patient comprising

identifying a patient suffering from cancer or at risk of cancer and

administering a membrane potential regulating agent to the patient in an amount effective to hyperpolarize malignant cells.

### BRIEF DESCRIPTION OF THE DRAWINGS

FIG. 1: Hepatocyte membrane potentials (PDs) in four malignant human cell lines and cultured, non-malignant human hepatocytes. Non-malignant hepatocytes were derived from patients undergoing surgical resections for solitary metastases to the liver. Results represent the mean±SD of 4-6 determinations. The differences between each malignant cell line and non-malignant hepatocytes were significant at p values<0.0001.

FIG. 2: RT-PCR for human GABA_{A}-β₃ receptor mRNA expression in four malignant human cell lines.

FIG. 3: Western blot analysis for GABA_{A}-β₃ receptor protein expression in four malignant human cell lines.

FIG. 4: RT-PCR for human GABA_{A}-ε receptor mRNA expression in the four malignant cell lines.

FIG. 5: RT-PCR for human GABA_{A}-β₃ receptor mRNA expression following stable transfection with GABA_{A}-β₃ receptor cDNA in a pcDNA 3.1 vector/V5-His C vector.

FIG. 6: Western blot analysis for human GABA_{A}-β₃ receptor protein expression following stable transfection with GABA_{A}-β₃ receptor cDNA in a pcDNA 3.1 vector/V5-His C vector.

FIG. 7: Representative DNA analysis by FACScan of Chang cells transfected with GABA_{A}-β₃ receptor cDNA or vector alone. In this scan, the percent of cells in G₀/G₁ (M1) and G₂/M phases (M2) of the cell cycle were 61% and 19% respectively in vector transfected cells (upper panel) and 42% and 25% respectively in GABA_{A}-β₃ receptor cDNA transfected cells (lower panel).

FIG. 8: Growth curves for Chang cells transfected with GABA_{A}-β₃ receptor cDNA or vector alone over a 10 day culture period. (*p<0.01 and **p<0.001).

FIG. 9: Number of colonies growing in soft agar following inoculation (0.5×10⁴ cells) with Chang cells transfected with GABA_{A}-β₃ receptor cDNA or vector (p<0.0005).

FIG. 10: Representative photomicrograph of Chang cells transfected with GABA_{A}-β₃ receptor cDNA (left) or vector alone (right). There were 50% fewer abnormal mitoses in Chang cells transfected with GABA_{A}-β₃ receptor cDNA (9% versus 18% respectively).

FIG. 11: Effect of the GABA_{A} receptor agonist (Muscimol 50 μM) on membrane potentials (PDs) of Chang cells, Chang cells transfected with GABA_{A}-β₃ receptor cDNA and Chang cells transfected with vector alone. Only Chang cells transfected with GABA_{A}-β₃ receptor cDNA became more polarized following exposure to Muscimol (*p<0.01).

FIG. 12: Effect of increasing concentrations of Muscimol on the proliferative activity of Chang cells transfected with GABA_{A}-β₃ receptor cDNA or vector alone. Proliferative activity was significantly decreased in a dose dependent fashion in GABA_{A}-β₃ receptor cDNA transfected cells (*p<0.0001).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “PD” refers to transmembrane electrical potential difference. To calculate PDs the Nernst equation was used:

PD=−(RT/ZF)λn[Fᵢₙ dFₒᵤₜ)/(Fₒᵤₜ dFᵢₙ),

wherein fluorescence within the cell was designated intracellular fluorescence (Fᵢₙ) and fluorescence outside the cell as extracellular fluorescence (Fₒᵤₜ), PD is membrane potential, Z is the charge of the permeable ion, F is Faraday's constant, R is the ideal gas constant and T is the absolute temperature.

It is of note that PDs can also be determined microelectrode cell impalements and patch clamping.

As used herein, “hyperpolarizing” refers to an increase (i.e. more negative) in PD.

As used herein, “depolarizing” refers to a decrease (i.e. less negative) in PD.

As used herein, the term “treating” in its various grammatical forms refers to preventing, curing, reversing, attenuating, alleviating, minimizing, suppressing or halting the deleterious effects of a disease state, disease progression, disease causitive agent other abnormal condition.

As used herein, “membrane potential regulating agent” refers to chemical and/or biological agents known in the art for altering cell membrane potential or cell membrane electrical charge. These include GABA receptor agonists, for example, Muscimol, Valproate, and Baclophen; barbiturates, for example, phenobarbital, primidone, and thiopental; benzodiazepenes, for example, diazepam, lorazepam and clonazepam; GABA antagonists (which often may also act as agonists), for example, fluroquinolones, bicuculline and Picrotoxin; sodium channel antagonists, for example, valproate, lamotrigine, carbamazepine, phenytoin, propafenone, and quinidine; potassium channel agonists, for example cromakalin; adrenergic agents, for example beta blockers (eg. propranonol); and glucagon. As will be appreciated by one knowledgeable in the art, these examples are provided for illustrative purposes only and are by no means limiting as any suitable membrane potential regulating agent may be used.

“Therapeutically effective amount” refers to the amount of a pharmaceutical compound required to have the desired effect.

As will be appreciated by one knowledgeable in the art, disorders or conditions having undesirable cell proliferation include, for example, some forms of cancer, for example, liver cancer, thyroid cancer, cervical cancer, breast cancer and gastric cancer, scar tissue formation and psoriasis.

Cell membranes have a specific electrical charge that can be altered by various means known in the art including drugs and introducing or changing the expression/activity of certain membrane receptors. We have found that by changing the electrical charge of cell membranes we can influence cell growth. For example, by depolarizing hepatocytes we can stimulate cell growth and by hyperpolarizing hepatocytes, we can inhibit cell growth. We have found that these effects can be achieved in malignant cells. This is in contrast with the prior art, which teaches that cell membrane potentials reflect intracellular metabolic events and does not teach that cell membrane potential influences cell growth. The purpose of the present study was to test the hypothesis that malignant hepatocytes are permanently depolarized as a result of downregulated or absent GABAergic activity. As a corollary to this hypothesis, we proposed that by restoring hepatocyte PDs towards those of resting hepatocytes (via increasing GABAergic activity) the proliferative and malignant features of these cells would revert to those associated with non-malignant hepatocytes.

As will be appreciated by one knowledgeable in the art, it is desirable to stimulate cell growth when growth is required as in regeneration of, for example, liver, heart or brain tissue following cell death or trauma. It is also desirable to inhibit cell growth when cells are malignant or are undergoing undesirable proliferation, for example, some forms of cancer.

Previously, stimulation of cell growth has been attempted by applying or enhancing the effect of growth promoters such as hormones or by inhibiting growth inhibitors. It is of note that the growth stimulators known in the art show either no or only limited growth stimulation and also have obvious potential side effects. Inhibition of cell growth has been achieved by chemotherapeutic agents, radiation therapy and/or immune mechanisms. These methods often do not completely inhibit the malignant growth and also have considerable side effects, such as damage to adjacent tissues.

Hepatocyte proliferation is a complex process that not only involves the levels of various growth promoters and inhibitors but also where those regulators are located within the cell (Steer, 1995, FASEB J 9: 1396-1400). Differences in electrical gradients across cell membranes may be responsible for determining their intracellular distribution.

Resting hepatocytes have a carefully regulated transmembrane electrical potential difference (PD) of approximately −35 mV (Wondergem and Harder, 1980, Journal of Cellular Physiology 102: 193-197). In previous studies we documented that following a growth stimulus such as 70% partial hepatectomy, hepatocytes promptly depolarize to PD values of approximately −20 mV (Zhang et al., Hepatology 23: 549-551). This depolarized state remains in effect until hepatocyte proliferative activity wanes. It is also of note that maintenance of hepatocyte PD at resting levels interferes with hepatocyte proliferative activity (Minuk et al., 1997, Hepatology 25: 1123-1127).

Numerous channels, pumps, receptors and the agents influencing the activity of these sites contribute to the PD of hepatocytes (Fitz and Scharschmidt, 1987, Am J Physiol 252: G56-G64). Recently, we identified specific GABA receptors on the surface of isolated rat and human hepatocytes that regulate chloride flux (Erlitzki et al., 2000, Am J Physiol 279: G738-G739). Activation of these sites with GABA_{A}receptor agonists caused prompt hepatocyte hyperpolarization and inhibition of proliferation, whereas exposure to GABA_{A} receptor antagonists depolarized hepatocytes and enhanced proliferative activity (Minuk et al., 1987, Am J Physiol 252: G642-G647; Zhang et al., 1998, J Hepatol 209: 638-641; Kaita et al., 1998, Hepatology 27: 533-546). These data indicate that the GABAergic system is an important regulator of hepatocyte PD and proliferative activity.

As discussed above, the methods currently used to stimulate or inhibit cell growth are often less effective than desired and also have potentially serious side effects. As discussed above, the prior art teaches that cell membrane potential reflects intracellular metabolism and has no role in or effect on cell growth. However, it is shown herein that by altering cell membrane charge, it is possible to either inhibit or induce cell growth. As will be apparent to one knowledgeable in the art, means known in the art for manipulating cell membrane potential or cell membrane electrical charge can be used as membrane potential regulating agents under appropriate conditions to either induce or inhibit cell growth.

For example, a therapeutically effective amount of a membrane potential regulating agent may be used for treating diseases characterized by undesired growth, for example, liver cancer, thyroid cancer, cervical cancer, breast cancer, gastric cancer, psoriasis or scar tissue formation. In these embodiments, the membrane potential regulating agent is injected or targeted to the vicinity of the tissue exhibiting undesired growth. Therein, the membrane potential growth regulating agent will polarize the membranes of cells in the vicinity, thereby slowing growth, as discussed below.

In some embodiments, the membrane potential regulating agent comprises an expression vector encoding GABA receptor, and the expression vector is introduced into the tissue of interest such that an amount of GABA receptor effective to hyperpolarize malignant cells is expressed in the patient.

In yet other embodiments, the membrane potential regulating agents may be administered in the vicinity of a tissue of interest in a therapeutic amount suitable to depolarize cell membranes, thereby inducing growth of cells within the tissue. This may be used, for example, for regenerating damaged tissue, for example, liver, kidney, heart or brain tissue. It is further of note that this administration may take place either in vitro or in vivo.

Examples of suitable membrane potential regulating agents include GABA receptor agonists, for example, Muscimol, Valproate, and Baclophen; barbiturates, for example, phenobarbital, primidone, and thiopental; benzodiazepenes, for example, diazepam, lorazepam and clonazepam; GABA antagonists (which often may also act as agonists), for example, fluroquinolones, bicuculline and Picrotoxin; sodium channel antagonists, for example, valproate, lamotrigine, carbamazepine, phenytoin, propafenone, and quinidine; potassium channel agonists, for example cromakalin; adrenergic agents, for example beta blockers (eg. propranonol); and glucagon. As will be appreciated by one knowledgeable in the art, these examples are provided for illustrative purposes only and are by no means limiting as any suitable membrane potential regulating agent may be used.

As discussed above, therapeutically effective amounts of these pharmaceutical compositions can be used to treat conditions wherein uncontrolled or undesirable cell proliferation is occurring,. In other embodiments, the pharmaceutical compositions are used to promote cell growth for regeneration, for example, brain, heart or liver tissue. As will be appreciated by one of skill in the art, the amount of agent required will vary according to patient age, weight, condition, and symptom severity as well as a number of other factors. In some embodiments, a concentration of approximately 1 μM to 1 mM of the membrane potential regulating agent may be used.

The membrane potential regulating agent or combinations thereof may be combined with any suitable carrier, excipient or binder known in the art. In other embodiments, the membrane potential regulating agent may be combined with permeation enhancers. In yet other embodiments, the membrane potential regulating element may be arranged to be localized in a specific area, for example, combined with an adhesive or targetting agents. In other embodiments, the membrane potential regulating element may be arranged to be taken as a pill or capsule.

In some embodiments, the membrane potential regulating agent at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

The invention provides kits for carrying out the methods of the invention. Accordingly, a variety of kits are provided. The kits may be used for any one or more of the following (and, accordingly, may contain instructions for any one or more of the following uses): treating liver cancer, thyroid cancer, cervical cancer, breast cancer, gastric cancer or psoriasis in an individual; preventing or inhibiting rapid cell or tissue growth in an individual at risk of liver cancer, thyroid cancer, cervical cancer, breast cancer, gastric cancer or psoriasis; preventing one or more symptoms associated with rapid cell or tissue growth or the like in an individual at risk of liver cancer, thyroid cancer, cervical cancer, breast cancer, gastric cancer or psoriasis; reducing severity one or more symptoms or rapid cell or tissue growth in an individual at risk of liver cancer, thyroid cancer, cervical cancer, breast cancer, gastric cancer or psoriasis; reducing recurrence of one or more symptoms associated with rapid cell or tissue growth in an individual at risk of liver cancer, thyroid cancer, cervical cancer, breast cancer, gastric cancer or psoriasis; suppressing rapid cell or tissue growth in an individual at risk of liver cancer, thyroid cancer, cervical cancer, breast cancer, gastric cancer or psoriasis; delaying development of rapid cell or tissue growth and/or a symptom of in an individual at risk of liver cancer, thyroid cancer, cervical cancer, breast cancer, gastric cancer or psoriasis; reducing duration of rapid cell or tissue growth in an individual at risk of liver cancer, thyroid cancer, cervical cancer, breast cancer, gastric cancer or psoriasis.

In other embodiments, the kits may be used for any one or more of the following (and, accordingly, may contain instructions for any one or more of the following uses): promoting tissue regeneration in an individual; preventing or inhibiting tissue necrosis in an individual having damaged or diseased brain, heart, kidney or liver tissue.

The kits of the invention comprise one or more containers comprising at least one membrane potential regulating agent, a suitable excipient as described herein and a set of instructions, generally written instructions although electronic storage media (e.g., magnetic diskette or optical disk) containing instructions are also acceptable, relating to the use and dosage of the membrane potential regulating agent for the intended treatment. The instructions included with the kit generally include information as to dosage, dosing schedule, and route of administration for the intended treatment. The containers of the membrane potential regulating agent may be unit doses, bulk packages (e.g., multi-dose packages) or sub-unit doses.

Referring to the figures, it is shown therein that transformation of malignant cells with GABA receptor results in normal mitotic division, compared to the abnormal mitotic division observed in transformed control cells.

The invention will now be described by way of examples. However, the invention is not limited to the examples.

As will be appreciated by one of skill in the art and as discussed below, the PD may vary according to cell type, how it is measured and under what conditions it is measured. For illustrative purposes, an isolated hyper-polarized hepatocyte has a PD of approximately −30 to −35 mV; an isolated normal hepatocyte has a PD of approximately −15 to −25 mV; and an isolated depolarized hepatocyte has a PD of approximately −3 to −10 mV.

Polyamines and the Liver

The polyamines which include putrescine, spermidine and spermine, are polyvalent oliphatic cations that are ubiquitous in mammalian cells (Pegg and McCann, 1982, Am J Physiol 243: C212-C221). Their importance in enhancing hepatocyte proliferation has been emphasized in studies documenting that in the presence of difluromethylornithine (DFMO) an inhibitor of ornithine decarboxylase (ODC), the rate limiting enzyme responsible for polyamine synthesis, hepatic regenerative activity following partial hepatectomy is minimal and not restored to normal until exogenous polyamines are administered (Luk, 1986, Gastroenterology 90: 1261-1267). Moreover, ODC activity increases in proportion to the regenerative stimuli as do hepatic tissue levels of the polyamines themselves (Russell and Snyder, 1969, Mol Pharmacol 5: 253-262; Minuk et al., 1990, Hepatology 12: 542-546). Finally, putrescine therapy has been reported to be effective in the treatment of both fulminant and acute forms of liver disease where hepatic regeneration is impaired and tissue polyamine levels are low (Nishiguchi et al., 1990, Hepatology 12: 348-353; Diehl et al., 1990, Hepatology 12: 633-637).

The mechanism whereby polyamines contribute to cell proliferation in general and hepatocyte proliferation in particular, is thought to relate to the polyvalent properties of the compounds (Janne et al., 1991, Ann Med 23: 241-259). Following partial hepatectomy, polyamines migrate from the cytoplasm to the nucleus where they bind to nucleosomes resulting in helical twists and configurational changes to DNA that are necessary for DNA replication and transcriptional activator- and growth promoter-induced RNA transcription (Janne et al., 1991; Haddox and Russell, 1981, PNAS USA 78: 1712-1716). Polyamines also bind to unstablized mRNA and tRNA resulting in enhanced transcriptional and translation activity (Poso and Raina, 1978, Biochim Biophys Acta 473: 241-293).

We have reported that following partial hepatectomy, hepatocyte membrane potentials promptly depolarize from resting membrane potentials (PD) of approximately −35 mV to PD values of approximately −25 mV (Minuk et al., 1997, Hepatology 25: 1123-1127). Such depolarization would be permissive to the positively charged polyamines migrating from the cytoplasm to the negatively charged DNA of the nucleus. We have also demonstrated that the depolarized state (which appears to be regulated by the expression of gamma aminobutyric acid (GABA)-receptors) persists for only that period of time during which active DNA synthesis occurs (1824 h) (Minuk et al., 1997; Zhang et al., 1996, Hepatology 23: 549-551; Minuk et al., 1987, Am J Physiol 252: 642-647).

GABA and the Liver

GABA is a monocarboxylic amino acid derived from the oxidation of putrescine. In the liver, sodium-independent, bicuculline-sensitive GABA_{A} receptor sites have been identified (Minuk et al., 1987). GABA_{A} receptors largely exist as pentameric structures consisting of various combinations of five major receptor sub-types: α, β,γ, δ and ρ (Stephenson, 1995, Biochem J 310: 1-9). Within four of these sub-units, the following isoforms have been identified: α1-α6, β1-β3, γ1-γ3, and ρ1-ρ2. The most ubiquitously expressed sub-units are α1, α2, α3, β2, β3 and γ2. In both the brain and liver, GABA binding to its receptors is regulated by a sodium-dependent, bicuculline insensitive, GABA transport protein (GABA-TP) which serves to maintain GABAergic activity at constant levels (Nelson and Blaustein, 1982, J Membrane Biol 69: 213-223; Herbison, 1997, Brain Research Bulletin 44: 321-326). Thus, when GABA_{A} receptor activity is transiently increased, GABA-TP expression increases (resulting in additional clearance of GABA from the sinusoidal space), whereas when GABA_{A}receptor activity is transiently decreased, GABA-TP expression decreases (resulting in decreased clearance of GABA from the sinusoidal space).

Activation of GABA_{A} receptor sites results in increases in chloride ion influx and maintenance of cells in a hyper-polarized state (Minuk et al., 1987; Stephenson, 1995; Nelson and Blaustein, 1982). During hepatic regeneration, GABAergic activity is attenuated and hepatocytes depolarize (Zhang et al., 1996; Minuk, 1993, Dig Dis 11: 45-54). Restoration of GABAergic activity restores membrane potentials to their hyperpolarized state and in the process, inhibits hepatic regeneration (Minuk et al., 1997; Minuk and Gauthier, 1993, Gastroenterology 104: 217-221). Of clinical relevance are findings that in acute and chronic forms of liver disease where serum GABA levels are increased and hepatic regenerative activity is impaired, GABA_{A}receptor antagonists enhance hepatic regeneration and improve clinical outcome (Zhang et al., 1995, Hepatology 22: 1797-1800; Kaita et al., 1998, Hepatology 27: 533-536; Zhang et al., 1996, Gastroenterology 110: 1150-1155).

Polyamines/GABA and HCC

Various groups have suggested that polyamines play an important role in the pathogenesis of cancer (Janne et al., 1978, Biochim Biophys Acta 473: 241-293). Perhaps most compelling is a study by Auvinen et al who reported that NIH 3T3 cells transfected with expression vectors containing human ODC cDNA, acquired growth and transforming properties consistent with malignant cells (Auvinen et al., 1992, Nature 360: 355-358). In addition, the transformed cells proliferated more rapidly than untransfected and ODC antisense construct controls. Other supportive data include reports of: increased ODC and adenosylmethionine decarboxylase activity (the latter enzyme being responsible for putrescine conversion to spermidine) in animal models of HCC (Don and Bachrach, 1975, Cancer Res 35: 3618-3622; Tamori et al., 1994, Hepatology 20: 1179-1186), increases in putrescine and spermidine concentrations in HCC when compared to adjacent non-tumor tissues (Janne at al., 1991; Tamori et al., 1994), and the beneficial effects of DFMO on tumor growth that can be reversed by exogenous polyamine administration (Sunkara et al., 1987, in Inhibition of Polyamine Metabolism: Biological Significance and Basis for New Therapies, McCann et al. Eds (San Diego: Academic Press)). In addition to their own intrinsic growth promoting properties, polyamines also facilitate and enhance the tumor promoting effects of other growth promoters (Poso and Raina, 1978; Manni et al., 1990, Breast Cancer Res Treat 17: 187-196). For example, in certain breast cancer clones, inhibition of putrescine synthesis with DMFO results in a complete blockage of the growth promoting effects of TGF-α (Kim et al., 1991, Breast Cancer Res Treat 18: 83-91). Similarly, the transfected C-HA-rase oncogene increases ODC mRNA as well as ODC enzyme synthesis and stability (Holtta et al., 1988, JBC 263: 4500-4507). Taken together, the above data point to increased polyamine activity contributing to the pathogenesis or at least being permissive towards, rather than an effect on hepatocyte carcinogenesis.

That abnormalities of the GABAergic system contribute to the pathogenesis of HCC is supported by the following data; (1) enhanced GABAergic activity results in termination of squamous cell carcinoma growth in mice as well as growth inhibition of various transformed cell lines (Boggust and Al-Nakib, 1986, IRCS Med Sci 14: 174175; Cerino et al., 1985, J Immunol Methods 77: 229-235; Tsutsumi et al., 1994, Gastroenterology 107: 499-504; Leder and Leder, 1975, Cell 5: 319-322; Langdon et al., 1988, Cancer Res 48: 6161-6165); (2) newly developed anti-tumor agents such as acivicin increase localized GABA content by as much as 140% (McGovern et al., 1989, Res Commun Chem Pathol Pharmacol 63: 215-229); (3) the majority of tumors studied to date are depolarized relative to adjacent non-tumor tissue, a finding in keeping with decreased GABAergic activity (Marino et al., 1994, Tumor Biol 15: 8289); (4) exposure to GABA_{A} receptor agonists inhibits the proliferation of malignant hepatocytes transfected with sub-units of the GABA_{A} receptor gene (Zhang et al., 2000, J Hepatol 32: 85-91); (5) enhancers of GABA_{A} receptor activity such as ethanol inhibit HCC growth (Shiina et al., 1990, AJR 155: 1221-1226) and (6) GABAergic activity as reflected by GABA-TP mRNA expression is decreased by 90% in human HCC compared to adjacent cirrhotic non-HCC and healthy liver tissues (Gong et al., 1997, Hepatology 26: 131A).

From the above, it is proposed that malignant hepatocytes have decreased or absent GABAergic activity that results in a permanent state of cell depolarization. The depolarized state permits the constant migration of polyamines from the cytoplasm to the nucleus where they enhance DNA synthesis and facilitate the transcription of other growth factors/carcinogens that promote DNA replication. A corrolary to this hypothesis which has significant clinical implications is that restoration of malignant hepatocyte membrane potentials to those associated with a non-malignant, resting state, should result in an inhibitory effect on HCC development and growth.

Materials

Dulbecco's modified Eagle's medium (DMEM), pyruvate, penicillin, streptomycin, geneticin (G-418) and Fungizone were purchased from GIBCO/BRL (Life Technology, Burlington, ON). Cool Calf and muscimol were purchased from Sigma (Sigma, Co., St. Louis, Mo.). Chang, Hep G₂ and PLC/PRF/5 cells were obtained from ATCC (America type Culture collection, Bethesda, Mass.). Healthy human hepatocytes were isolated from normal segments of liver tissues derived from patients undergoing surgical resections for benign hepatic lesions or solitary metastatic tumors to the liver as described by Roberts et al (Roberts et al., 1994, Hepatology 19: 1390-1399). The pcDNA3.1/V5-His C vector was purchased from Invitrogen (Carlsbad, Calif.) while the plasmid pRK5-beta3 containing the GABA_{A}-β₃ receptor gene was kindly provided by Professor P. Seeburg, University of Heidelberg, Heidelberg, Germany.

Cell Culture

Cells were grown in DMEM and supplemented with 10% cool calf serum, 1% penicillin (10,000 units/ml)/streptomycin (10,000 pg/ml), 1% fungizone, and 0.011% sodium pyruvate in a humidified, 37° C. incubator in an atmosphere of 95% air and 5% CO₂.

Cell PD Determinations

A Leica DM IRB fluorescence microscope equipped with a PDMI-2 Open perfusion Micro-incubator (Harvard Apparatus, Saint-Laurent, Quebec) was used to measure PDs as described by Loew (Loew, 1998, Cell Biology: A Laboratory Handbook). The incubating temperature was 37° C. Twenty four hour cultured healthy, human hepatocytes and malignant hepatocytes were washed with NB buffer (130 mM NaCl, 5.5 mM KCl, 1.8 mM CaCl₂, 1.0 mM MgCl₂, 25 mM glucose and 20 mM HEPES buffer adjusted to pH 7.4) three times and then incubated for 10 min with NTB buffer (100 nM tetramethylrhodamine ethyl ester (TMRE) in NB buffer). After obtaining photographs of the fluorescent cells, they were washed with KB buffer (130 mM KCl, 5.5 mM NaCl, 1.8 mM CaCl₂, 1.0 mM MgCl₂, 25 mM glucose and 20 mM HEPES buffer adjusted to pH 7.4) three times and incubated for 10 min with DTB buffer (100 nM TMRE and 1 μM valinomysin in KB buffer). Photographs were then obtained of the depolarized cells. Fluorescence within the cell was designated intracellular fluorescence (Fᵢₙ) and fluorescence outside the cell as extracellular fluorescence (Fₒᵤₜ). To calculate PDs the Nernst equation was used.

PD=−(RT/ZF)λn[Fᵢₙ dFₒᵤₜ)/(FₒᵤₜdFᵢₙ),

where PD is membrane potential, Z is the charge of the permeable ion, F is Faraday's constant, R is the ideal gas constant and T is the absolute temperature.

RT-PCR for GABA_{A}-β₃ Receptor

Total RNA was extracted from 1×10⁶ cells by the commercially available Trizol method (Invitrogen, Carlsbad, Calif.). 20 ul of reverse transcription reactions consisted of the following: 1 ug RNA, 5× reaction buffer (Clontech, Palo Alto, Calif., USA), dNTP 0.5 mM, RNase inhibitor 0.5 U, oligo (dT)18 primer 20 pmol, and MMLV reverse transcriptase 20 U. Reactions were incubated at 42° C. for 60 min, and terminated at 99° C. for 5 min. 5 ul of the reactions were used for the PCR reaction.

The oligonucleotide primers for PCR reaction were designed against human GABA_{A} receptor sequences using an Oligo 5.0 program (NBI, National Biosciences Inc., Plymouth, Minn., USA). The sequences of human GABA_{A}-β₃ receptor oligonucleotide primers were as follows: Forward primer, 5′ AAGGGCTGGTTACCGGAGTGGA 3′; Reverse primer, 5′ CGAAGATGGGTGTTGATGG 3′. The PCR amplification was carried out in 30 cycles of denaturation (94° C., 45 sec), annealing (57° C., 45 sec), elongation (72° C., 2 min) and with an additional 7 min final extension at 72° C. Finally, 10 ul of the PCR products were run on 2% agarose gels. The product length is 290 bp (Erlitzki et al., 2000).

SDS-Gel Electrophoresis and Immunoblotting Techniques

Cells were harvested by scraping into a protease inhibitor mixture consisting of 20 mM Tris (pH 7.4), 1 mM phenylmethylsulfonyl fluoride, 1 mM benzamidine, 100 uM leupeptin, and 5 mM EDTA and passed through a 26-gauge needle. Protein concentrations were measured using the Lowry protein assay (Lowry et al., 1951, J Biol Chem 193: 265-275). 50 μg of total protein extracts were separated on 12% polyacrylamide-SDS gels and electroblotted to nitrocellulose membranes as described previously (Erlitzki et al., 2000). Membranes were blocked with 5% skim milk in Tris-buffered saline (TBS, 0.02 M Tris-base, pH 7.6) for 1 hour at room temperature and incubated with rabbit anti-human GABA_{A}-β₃ receptor antibody (5.55 ug/ml) (provided by Dr. Werner Sieghart, University of Vienna, Austria) overnight at 4 ° C. Bands were detected with a horseradish peroxidase-labeled secondary antibody-catalyzed chemiluminescence reaction (ECL, Amersham Pharmacia Biotech, Burlington, ON) (Erlitzki et al., 2000).

Cell Proliferation

[³H]-thymidine incorporation was determined as described by Luk (Luk, 1986, Gastroenterology 90: 1261-1267). 5×10⁴ cells were seeded into 6-well plates. After two days of culture to allow attachment to plate bottoms, cells were incubated with 1 μCi [³H]-thymidine for 2 hours at 37° C. [³H]-thymidine incorporated into cellular DNA was precipitated by the addition of 10% trichloroacetic acid (TCA) for 15 min at room temperature. Cells were rinsed and resolublilized in a 0.3 M NaOH and 1% SDS solution then assayed for radioactivity in a y-scintillation counter.

Bromodeoxyuridine (BrdUrd) incorporation was determined by seeding 5×10³ cells into 96 well plates. At subconfluence, cells were incubated with serum-free DMEM. Twenty-four hours later, the media was switched to DMEM containing BrdUrd for 2 hours. Incoroporation of BrdUrd was determined using a 5-Bromo-2′-deoxyuridine Labeling and Detection Kit III (Roche Diagnostics, Mississauga, Ontario, Canada) according to the manufacturer's instructions. Absorbance at 450-490 nm was measured using a microplate reader (Molecular Devices, Menlo Park, Calif., USA).

WST-1 determinations were performed using a commercially available kit (Boehringer Mannheim Canada, Laval, Quebec, Canada). Briefly, 1×10⁴ cells were seeded into 96 well plates. After two days of culture one tenth the volume of 4-[3-(4-lodophenyl)-2-(4-nitrophenyl)-2H-5-tetrazolio]-1,3-benzene disulfonate (WST-1, Boehringer Mannheim, Laval, PQ) was added to each well, and the cells incubated with WST-1 for 4 h in 37° C. Asborbance at A₄₀₅-A₆₅₀ was determined by a microplate reader (Molecular Devices, Menlo Park, Calif., USA).

Colony formation in soft agar was conducted in 60 mm plates containing 2 layers of media. The top layer contained DMEM supplemented with 15% cold calf and 0.3% agarose while the bottom layer contained DMEM supplemented with 15% cold calf and 0.5% agarose. Chang cells and Chang cells transfected with GABA_{A}-β₃ or vector alone were harvested by trypsinization and 0.5×10⁴ cells were inoculated into the top layer of agarose. Triplicate plates were incubated at 37° C. under 5% CO₂, and the number of macroscopic colonies per plate were counted after 3 weeks of culture.

The number of cells undergoing mitoses was expressed as a percent of 100 cells counted per high power field (mitotic index). A total of six high power fields were examined per cell population. The identity of the cell population was not known to the cytologist (ER) calculating the mitotic index.

Plasmid Construction

The 1640-base pair cDNA containing the GABA_{A}-β₃ receptor coding region was cut with Xba I from plasmid pRK5-beta3 and cloned into the pcDNA3.1/V5-His C vector producing a plasmid referred to as pcDNA-beta3. The cloned cDNA fragment is under the transcriptional control of the immediate early gene of the human cytomegalovirus (CMV) promoter and the vector contains polyadenylation signals and ampicillin and zencin resistant genes (Sadji et al., 2000, Cell Signal 12: 745-750). pcDNA-beta3 plasmids were isolated, purified and sequenced to confirm that the cDNA of the GABA_{A}-β₃ receptor was in frame with the CMV promoter.

Stable Transfection

Briefly, 1×10⁶ cells in a 3.5-cm dish were transfected with 1 pg of linearized pcDNA-beta3 or pcDNA vector alone using Lipofectamine (GIBCO/BRL) according to manufacturer's instructions. Stable transfected cells were established in the presence of G418 (800 pg/ml), and resistant clones were isolated by using cloning cylinders and maintained under G418 selection (200 pg/ml). Clones were analyzed individually by RT-PCR and Western blotting for levels of GABA_{A}-β₃ receptor mRNA and protein expression respectively (Erlitzki et al., 2000).

DNA Analysis

1×10⁶ cells were stained with propidium iodide as described by Diez-Fernandez et al (Diez-Fernandez et al., 1993, Hepatology 18: 912-918). Emitted fluorescence was assayed in a FACScan flow cytometer (Becton-Dickinson). A double discriminator module was used to distinguish between single nuclei and nuclear aggregation.

Statistical Analyses

Analyses of variance followed by paired Student's t-tests for parametric data and a Wilcoxon Rank Sum test for non-parametric data were performed where appropriate. p-values<0.05 were considered significant. The results provided represent the mean+standard error of the mean unless otherwise indicated.

Cellular PDs

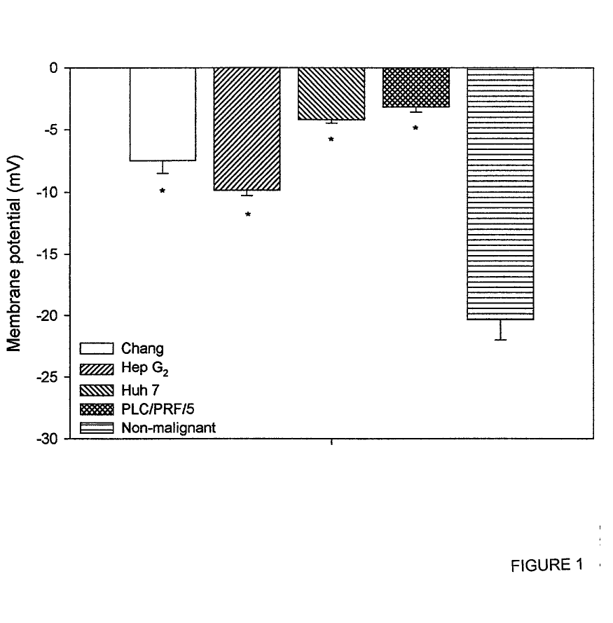

As shown in FIG. 1, relative to proliferating, non-malignant hepatocytes (PD; −20.1±1.6 mV), the PDs of all four malignant hepatocyte cell lines were markedly depolarized (Chang: −7.5±1.0, Hep G₂: −9.8±0.5, HuH-7: −4.2±0.3 and PLC/PRF/5: −3.2±0.4 mV, p<0.0001, respectively). The depolarized state was stable and did not fluctuate beyond 0.5-1.0 mV over a four-day period of repeated testing (data not shown).

GABA_{A} Receptor Expression

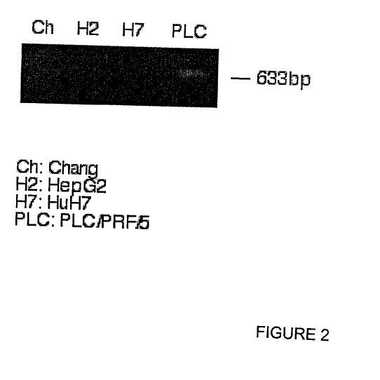

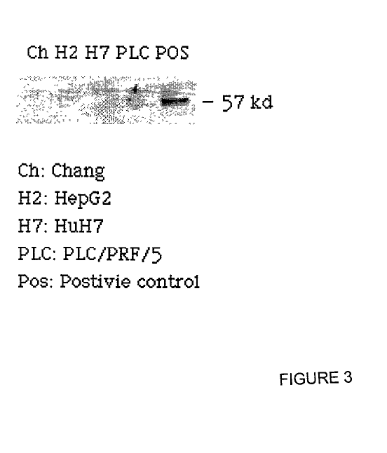

The human liver expresses two of the 15 known GABA_{A} receptor subtypes (β3 and ε) (Erlitzki et al., 2000; Stephenson, 1995, Biochem J 310: 1-9). Of the two, only β3 has the capacity to form GABA-gated channels (Stephenson, 1995). The results of RT-PCR for GABA_{A}-β3 receptor mRNA expression are shown in FIG. 2. In Chang, Hep G₂ and HuH-7 cells, expression was undetectable while in PLC/PRF/5 cells, expression was evident. Sequence analysis of the GABA_{A}-β3 receptor mRNA expressed in PLC/PRF/5 cells revealed a non-relevant, single mutation (G to A) at nt 1058. The absence of GABA_{A}-β3 receptor mRNA expression in the three receptor-deficient malignant cell lines was confirmed at the protein level by Western blot analyses. In the case of PLC/PRF/5 cells, a discordance existed in that despite the presence of the transcript, GABA_{A}-β3 receptor protein expression was absent by Western blot analysis (FIG. 3).

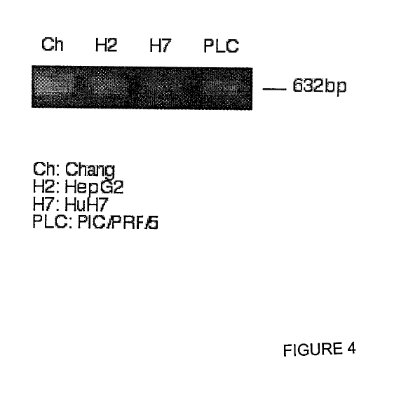

GABA_{A}-ε receptor mRNA and protein expression were present in all four malignant cell lines (FIG. 4).

Transfection Studies

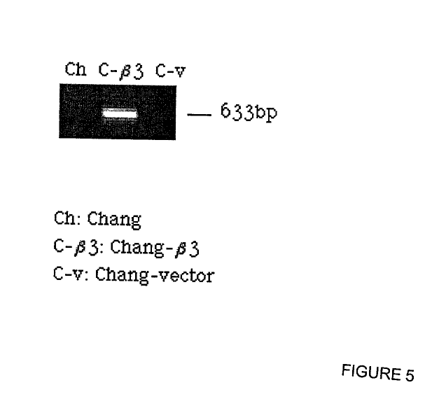

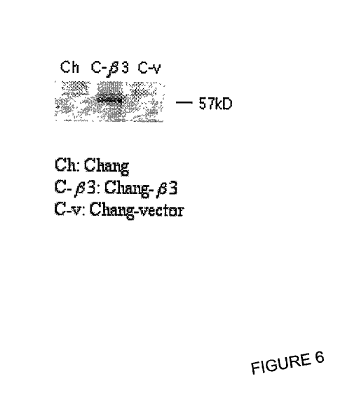

To determine what impact restoration of GABA_{A}-β3 receptor mRNA expression has on cellular PD and the proliferative activity of receptor-deficient cells, Chang cells were transfected with GABA_{A}-β3 receptor cDNA in pcDNA 3.1/V5-His C vector or vector alone. Restoration of GABA_{A}-β3 receptor mRNA and protein expression following transfection were confirmed by RT-PCR and Western blot analyses respectively but remained undetectable in cells transfected with vector alone (FIGS. 5 and 6). Following transfection, the PDs of GABA_{A}-β3 receptor cDNA transfected cells increased (became more hyperpolarized) from a baseline PD of −7.5±1.0 to a PD of 12.9±0.4 mV (p<0.0001) while the PDs of cells transfected with vector alone remained unaltered.

Proliferative Activity

S phase proliferative activity was documented by [³H]-thymidine and BrdUrd incorporation rates while M phase activity was documented by WST-1 activity, mitotic index and FACScan DNA analyses. In addition, cell doubling times and counts over a 10-day culture period were calculated. As shown in Table I, S phase activity was significantly decreased in GABA_{A}-β3 receptor cDNA-transfected Chang cells when compared to Chang cells transfected with vector alone. However, an even greater decrease was observed in M phase activity. Of note, the percent of GABA_{A}-β3 receptor cDNA-transfected cells undergoing mitosis after GABA_{A}-β3 receptor cDNA transfection (34%) was similar to that reported for non-malignant hepatocytes in culture (Diez-Fernandez et al., 1993).

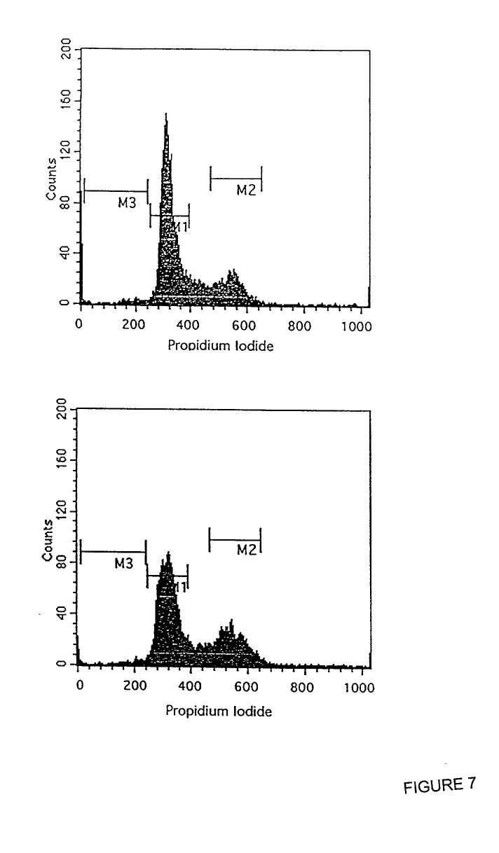

The results of DNA analyses by FACScan supported WST-1 and mitotic index findings. Specifically, the proportion of Chang cells transfected with GABA_{A}-β3 receptor cDNA in the G₀/G₁ phase of the cell cycle was decreased while those in the G₂/M phase were increased relative to Chang cells transfected with vector alone (FIG. 7).

The doubling time of Chang cells transfected with vector was 31.5±2.4 hours (Table I). In Chang cells transfected with GABA_{A}-β3 receptor cDNA the doubling time increased to 53.2±3.6 hours (p<0.0005).

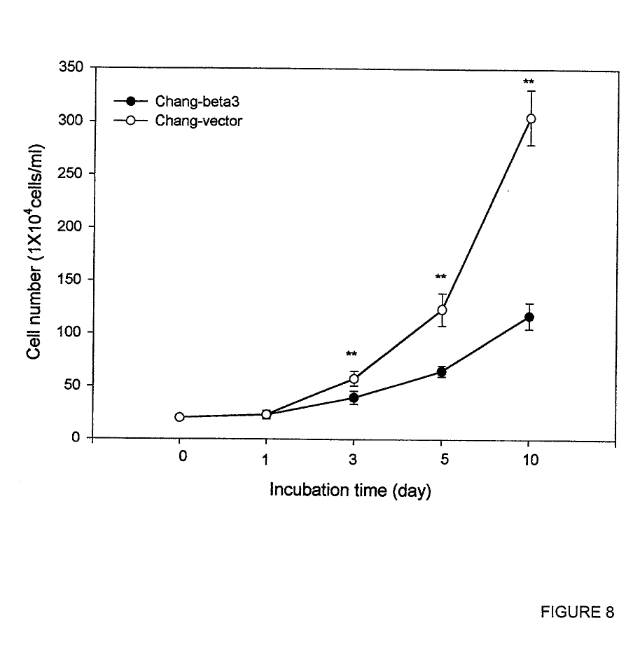

Over a 10-day period, Chang cells transfected with vector alone grew at exponential rates (FIG. 8). However, Chang cells transfected with GABA_{A}-β3 receptor cDNA grew at a slower rate such that by day 5, cell counts in GABA_{A}-β3 receptor cDNA-transfected cells were only 50% that of Chang cells transfected with vector alone and 30% on day 10 of culture.

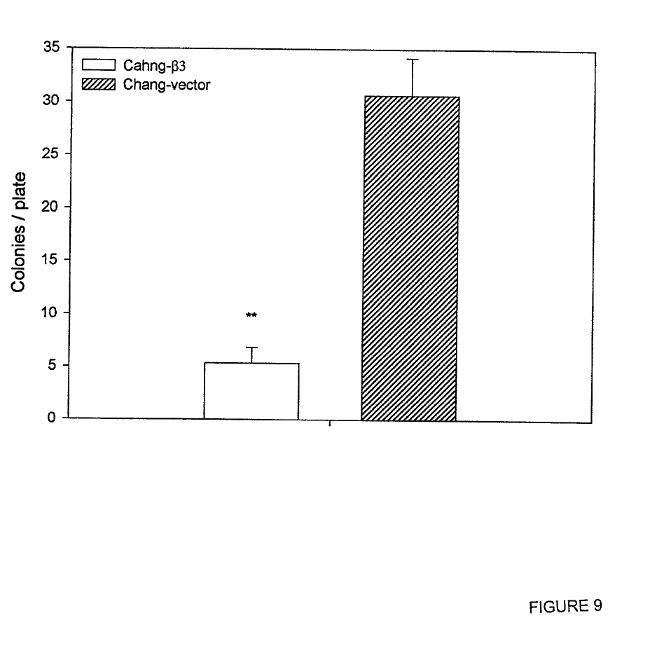

As shown in FIG. 9, colony formation in soft agar was significantly decreased in Chang cells transfected with GABA_{A}-β3 receptor cDNA when compared to cells transfected with vector alone (p<0.005).

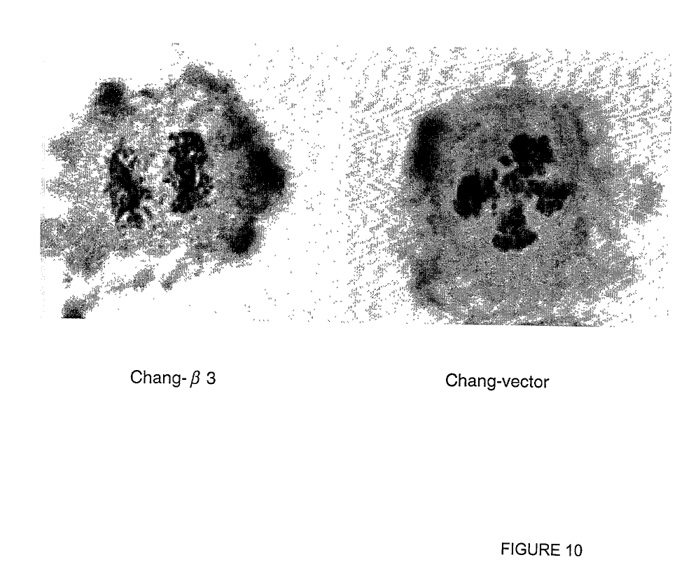

There were fewer abnormal mitoses (abnormal spindle patterns) in GABA_{A}-β3 receptor cDNA-transfected cells (9%) compared to Chang cells transfected with vector alone (18%). Chang cells transfected with vector alone also had more multinucleated cells with macronucleoli compared to those transfected with GABA_{A}-β3 receptor cDNA. Representative cells are shown in FIG. 10.

Receptor Augmentation

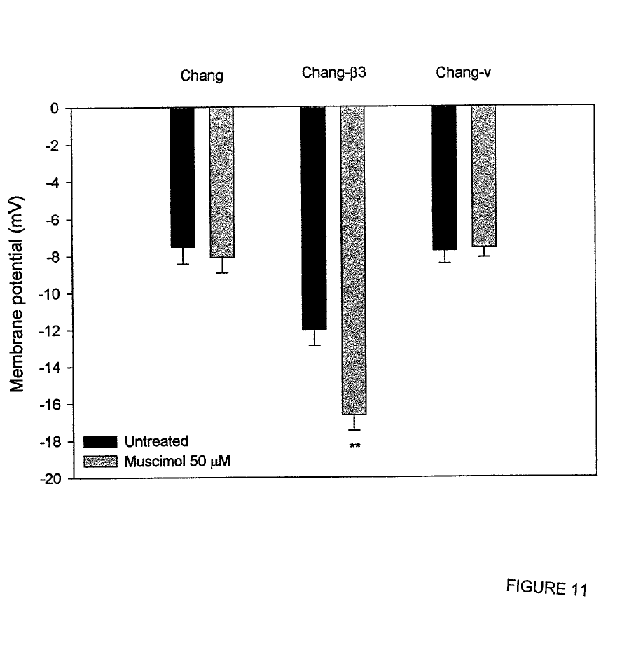

To determine whether further hyperpolarization could be achieved in Chang cells transfected with GABA_{A}-β3 receptor cDNA, these cells together with non-transfected Chang cells and Chang cells transfected with vector alone were exposed to 50 μM muscimol, a specific GABA_{A} receptor agonist for 48 hours. As shown in FIG. 11, muscimol had little or no effect on the PDs of non-transfected Chang cells and Chang cells transfected with vector alone but increased by approximately 40% the PDs of Chang cells transfected with GABA_{A}-β3 receptor cDNA (pre-muscimol: −12.0±0.9 versus post-muscimol: −16.4±0.8, p<0.01).

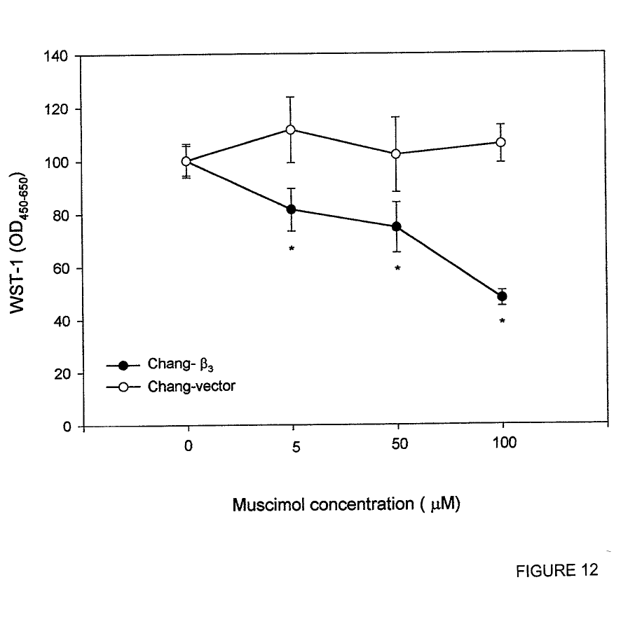

To determine whether the addition of muscimol and/or other GABA_{A} receptor agonists might have therapeutic value in inhibiting the growth of malignant hepatocytes with restored GABA_{A}β3 receptor mRNA expression, proliferative activity was documented in Chang cells transfected with GABA_{A}-β3 receptor cDNA or vector alone following exposure to varying concentrations of muscimol (0-100 μM). As shown in FIG. 12, a dose dependent decrease in proliferative activity was documented in GABA_{A}-β3 receptor cDNA-transfected cells while in Chang cells transfected with vector alone, proliferative activity remained essentially unaltered.

The results of this study indicate that malignant human hepatocyte cell lines are depolarized relative to cultured, non-malignant human hepatocytes. The results also indicate that GABA_{A}-β3 receptor mRNA expression is absent in at least some of these cell lines. As discussed above, it is of note that restoration of PD values towards those associated with resting, non-malignant hepatocytes results in a loss or attenuation of malignant features including decreased proliferative activity, slower growth rates, less colony formation in soft agar, and fewer numbers of cells with abnormal mitoses.

Other investigators have reported that malignant cells are significantly depolarized in situ and in cell culture systems (Marino et al., 1994, Tumor Biol 15: 8289). In the case of thyroid cancer, PDs of malignant thyroid cells were approximately 50% depolarized when compared to non-malignant thyroid cells (Jamakosmanovic and Loewenstein, 1968, J Cell Biol 38: 556-561). Similar extents of depolarization have been documented in glioma, cervical, breast and gastric carcinoma cells (Marino et al., 1994; Lash et al., 1955, Am J Obst Gynecol 70: 354-358; Picker et al., 1981, J Neurosurg 55: 347-363; Tokuoka and Morioka, 1957, Jpn J Cancer Res 48: 353-354). The only previous study describing PDs of malignant hepatocytes was reported by Binggoli and Cameron who documented PD values of −19.8±7.1 mV in rat hepatoma tissue compared to −37.1±4.3 mV in normal rat liver (Binggeli and Cameron, 1980, Cancer Res 40: 1830-1835). The higher PD values in that study reflecting the authors' use of in situ determinations compared to the isolated hepatocytes and cell lines employed in the present study (Moule and McGivan, 1990, Biochem Biophys Acta 1031: 383-397).

Hepatocytes derived from cirrhotic livers, which can be considered a potentially pre-malignant state, are depolarized when compared to hepatocytes derived from healthy livers (Burczynski et al., 1999, J Hepatology 30: 492-497). Furthermore, data described herein indicates that malignant features including [³H]-thymidine and BrdUrd incorporation rates, WST-1 activity, mitotic indices, doubling times, growth patterns, colony formation in soft agar, and the number of abnormal mitoses are significantly decreased and doubling times significantly prolonged when cells are hyperpolarized by augmenting GABA_{A} receptor expression.

Whereas PD determinations have been reported in various malignant cell lines and tissues, to our knowledge, direct determinations of GABA_{A} receptor expression have hitherto not been described. Nonetheless, our finding of absent GABA_{A}-β3 receptor mRNA expression was not unexpected as in a previous study, we described the absence and/or marked downregulation of the sodium dependent GABA transporter system in seven human HCC tissues and GABA-transporter expression tends to parallel GABA_{A} receptor activity (Gong et al., 1997, Hepatology 26: 131A; Herbison, 1997, Brain Research Bulletin 44: 321-326).

Despite the lack of documentation of GABA receptor expression in malignant cells and/or tissues, others have reported that augmentation of GABAergic activity, by the administration of high concentrations of GABA or GABA receptor agonists, has an inhibitory effect on cell proliferative activity (Boggust and Al-Nakib, 1986, IRCS Med Sci 14: 174-175; Cerino et al., 1985, J Immunol Methods 77: 229-235; Tsutsumi et al., 1994, Gastroenterology 107: 499-504; Leder and Leder, 1975, Cell 5: 319-322; Langdon et al., 1988, Cancer Res 48: 6161-6165; Tatsuta et al., 1992, Int J Cancer 52: 924-927; Tatsuta et al., 1990, Cancer Res 50: 4931-4934).

The results of our GABA_{A}-β₃ receptor cDNA-transfection experiments indicate the same effect can be achieved by restoring GABA_{A} receptor expression in malignant hepatocytes.

The findings of more pronounced inhibition of M phase (decreased WST-1 expression and a lower mitotic index) relative to S phase (decreased [³H]-thymidine and BrdUrd incorporation) activity in GABA_{A}-β₃ receptor transfected cells suggests that cell cycle arrest is occurring predominantly in the G₂ phase of the cell cycle. The G₂/M increase on FACScan supports this interpretation.

Not all malignant hepatocyte cell lines had absent GABA_{A}-β₃ receptor mRNA expression despite a uniformly depolarized state. Indeed, mRNA expression in PLC/PRF/5 cells was similar if not increased relative to healthy, human hepatocytes. That sequencing of the transcript failed to identify mutations resulting in functional changes at the cell membrane level suggests that other factors are responsible for the absence of GABA_{A}-β₃ receptor expression at the protein level.

The GABA_{A}-ε receptor is the only GABA_{A} receptor subtype that does not form GABA-gated chloride channels (Stephenson, 1995). Thus, unlike the β₃ receptor subtype, the ε receptor subtype has no inherent electrogenic properties. Recent data suggest its role is in regulating the distribution and display of other GABA_{A} receptor subtypes throughout the cell (Davies et al., 1997, Nature 385: 820-823).

Thus, the results of the present study teach new approaches to the treatment of several diseases, including, as discussed above, HCC. For example, interventions that result in tumor hyperpolarization would be expected to have an inhibitory effect on tumor growth. In the case of HCC, such interventions may require gene therapy with the GABA_{A}-β₃ receptor gene as the prevalence of receptor-deficient or mutated cells appears to be high—all four malignant hepatocyte cell lines in this study and four of four HCC tissues studied to date. GABA is rapidly cleared from the systemic circulation and presently available GABA_{A} receptor agonists have only a transient effect on receptor activity (Minuk et al., 1987; White and Sato, 1978, J Neurochemistry 31: 41-47; Ferenci et al, 1983, Hepatology 3: 507-512). Whether inhibition of hepatic GABA metabolism, interference with GABA clearance by hepatocytes, and/or manipulation of non-GABAergic electrogenic systems such an increasing Na+/K+ATPase activity can be exploited to achieve the desired PD changes remains to be determined (Moule et al., 1987, Biochem J 241: 737-743; Lee and Clemens, 1992, Am J Physiol 26: G319-G326). It should also be noted that augmentation of GABAergic activity in the liver inhibits healthy hepatocyte proliferation, an important survival mechanism in patients with advanced liver disease (Zhang et al., 1998; Kaita et al., 1998).

In conclusion, the results of this study indicate that malignant hepatocytes exist in a significantly depolarized state. This depolarized state is associated with absent or limited GABA_{A}-β₃ receptor expression. The results also indicate that increasing the PD of malignant hepatocytes by transfecting receptor-deficient cells with GABA_{A}-β₃ receptor cDNA, results in a loss or attenuation of malignant features.

While the preferred embodiments of the invention have been described above, it will be recognized and understood that various modifications may be made therein, and the appended claims are intended to cover all such modifications which may fall within the spirit and scope of the invention.

1. A method of regulating cell growth comprising:
administering an effective amount of a membrane potential regulating agent to a tissue portion.

2. The method according to claim 1 wherein the membrane potential regulating element depolarizes cell membranes, thereby inducing cell growth.

3. The method according to claim 1 wherein the membrane potential regulating element hyperpolarizes cell membranes, thereby inhibiting cell growth.

4. The method according to claim 1 wherein the membrane potential regulating agent is selected from the group consisting of a GABA receptor agonists, a barbiturate; a benzodiazepene; a GABA antagonist; a sodium channel antagonist; a potassium channel agonist; and an adrenergic agent.

5. The method according to claim 4 wherein the GABA receptor agonist is Muscimol, Valproate, or Baclophen.

6. The method according to claim 5 wherein the GABA receptor agonist is Muscimol.

7. The method according to claim 4 wherein the barbiturate is phenobarbital, primidone or thiopental.

8. The method according to claim 4 wherein the benzodiazepene is diazepam, lorazepam or clonazepam.

9. The method according to claim 4 wherein the GABA antagonist is fluroquinolones, bicuculline or Picrotoxin.

10. The method according to claim 4 wherein the potassium channel agonist is cromakalin.

11. The method according to claim 4 wherein the sodium channel antagonist is valproate, lamotrigine, carbamazepine, phenytoin, propafenone, or quinidine.

12. The method according to claim 4 wherein the adrenergic agent is a beta blocker or glucagon.

13. A method of treating a patient with cancer, comprising introducing into the patient an expression vector encoding GABA receptor, such that an amount of GABA receptor effective to hyperpolarize malignant cells is expressed in the patient.

14. The method according to claim 13 wherein the expression vector is introduced into the vicinity of cancerous tissue.

15. The method according to claim 13 wherein the cancer is liver cancer.

16. A method of treating cancer in a patient comprising identifying a patient suffering from cancer or at risk of cancer and administering a membrane potential regulating agent to the patient in an amount effective to hyperpolarize malignant cells.

17. The method according to claim 16 wherein the membrane potential regulating agent Is introduced into the vicinity of cancerous tissue.

18. The method according to claim 16 wherein the membrane potential regulating agent is selected from the group consisting of a GABA receptor agonists, a barbiturate; a benzodiazepene; a GABA antagonist; a sodium channel antagonist; a potassium channel agonist; and an adrenergic agent.

19. The method according to claim 16 wherein the cancer is liver cancer.
